(19) United States
(12) Patent Application Publication
(10) Pub. No.: US 2004/0199072 A1
(43) Pub. Date: Oct. 7, 2004

(54) Integrated electromagnetic navigation and patient positioning device

(76) Inventors: Stacy Sprouse, Brighton, CO (US); Brad Clayton, Superior, CO (US); Mark W. Hunter, Broomfield, CO (US)

Correspondence Address: HARNESS, DICKEY & PIERCE, P.L.C., P.O. BOX 828, BLOOMFIELD HILLS, MI, 48303, US

(21) Appl. No.: 10/405,068

(22) Filed: Apr. 1, 2003

(51) Int. Cl. A61B 5/05; A47B 13/00

(52) U.S. Cl. 600/424; 5/601

(57) ABSTRACT

A patient positioning device used to position a patient during a navigated medical procedure includes a contoured patient support and a portion of a navigation system. The contoured patient support positions the patient in a desired manner. The portion of the navigation system is integrated within the patient support, such that the navigated medical procedure may be performed in a substantially unobstructed manner.

### FIELD OF THE INVENTION

The present invention generally relates to patient positioning devices, and more specifically, to patient positioning devices that integrate and incorporate an electromagnetic navigation system to assist in performing medical procedures.

### BACKGROUND OF THE INVENTION

Image guided medical and surgical procedures utilize patient images obtained prior to or during a medical procedure to guide a physician performing the procedure. Recent advances in imaging technology, especially in imaging technologies that produce highly detailed, computer generated two, three and four-dimensional images, such as computed tomography (CT), magnetic resonance imaging (MRI), isocentric C-arm fluoroscopic imaging, and two, three, and four-dimensional fluoroscopes or ultrasounds have increased the interest in image guided medical procedures.

During these image guided medical procedures, the area of interest of the patient that has been imaged is displayed on a display. The surgical instruments that are used during this medical procedure are tracked and superimposed onto the display to show the location of the surgical instrument relative to the area of interest in the body.

Other types of navigation systems operate as image-less systems, where an image of the body is not captured by an imaging device prior to the medical procedure. In this type of procedure, the system may use a probe to contact certain landmarks in the body, such as the landmarks on the bone, where the system generates either a two-dimensional or three-dimensional model of the area of interest, based upon these contacts. In this way, when the surgical instrument or other object is tracked relative to this area, they can be superimposed on this model and illustrated on the display.

However, various types of navigation systems employed during the image guided or non-image guided medical or surgical procedure suffer from certain disadvantages. For example, existing optical image guided navigation systems are subject to line-of-sight issues. With an optical navigation system, a clear unobstructed path between the optical trackers and the optical reflectors or emitters on rigid and non-rigid instruments should be maintained. During different types of medical procedures, this unobstructed path, however, may be difficult to maintain. With electromagnetic type navigation systems, electromagnetic generators or receivers are generally positioned adjacent the patient area being navigated, sometimes referred to as patient or navigation space. Here again, however, positioning of these electromagnetic transmitters or receivers may sometimes be difficult due to all of the surrounding equipment typically encountered in an OR room.

Moreover, various types of medical procedures typically require positioning or support devices in order to position the patient properly during the medical or surgical procedure. For example, during spinal procedures, the patient is typically positioned prone on a spinal support frame in order to position the spinal anatomy in the correct orientation for most spinal procedures. In some instances, this positioning may also be adjustable to account for different spinal procedures or the patient's size. Other types of surgical procedures, such as orthopedic procedures also typically require patient positioning devices or supports to be used during the procedures. For example, during knee surgery, supports are used to typically elevate the calf region of the patient with the knee in full flexion to provide the correct orientation for the knee procedure. Other medical procedures also require patient positioning devices designed specifically for the size of the patient, as well as the procedure. Here again, however, by positioning the patient on a patient positioning device, which is generally located on or incorporated into an OR table, it again makes it very difficult to properly position a navigation system to be used during the procedure.

With electromagnetic type navigation systems, the electromagnetic generators or transmitters and the electromagnetic receivers also must be positioned at appropriate distances apart from one another. In this regard, in order for the generated fields from the coils to converge to create an accurate navigation space, there must be some separation between the electromagnetic generators and the area being navigated. Again, in typical OR environments, it may be difficult to provide such separation between the transmitters and the navigation space. Still further, with the electromagnetic type navigation system, it is desirable to have little or no metal between the transmitter and receiver configuration in order to eliminate as much interference as possible. Here again, in a typical OR environment there are various patient support devices that make it difficult to reduce interference.

It is, therefore, desirable to provide an integrated electromagnetic localization and patient positioning device, which substantially reduces or eliminates the above identified disadvantages and drawbacks. It is, therefore, an object of the present invention to provide such an integrated electromagnetic navigation and patient positioning device to assist in medical procedures.

### SUMMARY OF THE INVENTION

In accordance with the teachings of the present invention, a patient positioning device to position a patient during a navigated medical procedure is disclosed. The patient positioning device may be formed into any type of shape used to support a patient during any type of medical procedure.

In an embodiment, a patient positioning device to position a patient during a navigated medical procedure includes a contoured patient support and a portion of the navigation system. The contoured patient support is operable to position the patient in a desired manner. The portion of the navigation system is integrated within the patient support wherein the navigated medical procedure may be performed in a substantially unobstructed manner.

In another embodiment, a patient positioning system to position a patient during a navigated medical procedure includes a first contoured patient support and a second contoured patient support. The first contoured patient support includes a first shape and is operable to position the patient with said first shape. The first contoured patient support also defines a first recess. The second contoured patient support includes a second shape and is operable to position the patient with the second shape. The second contoured patient support defines a second recess where the first recess is substantially the same as the second recess, whereby a portion of the navigation system may be selectively received within each of the first and second recesses.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is an exemplary diagram of a navigation system employing the integrated electromagnetic navigation and patient positioning device according to the teachings of the present invention;

FIGS. 2a and 2b are diagrams representing undistorted and distorted views of a fluoroscopic C-arm imaging device;

FIG. 3 is a perspective view of an integrated electromagnetic navigation and patient positioning device used for spinal procedures;

FIGS. 4a-4c are side views of various shaped patient positioning devices of FIG. 3 for use in spinal procedures;

FIG. 5 is an exploded perspective view of another integrated electromagnetic navigation and patient positioning device where the electromagnetic generators are non-removably incorporated directly within the patient positioning device;

FIG. 6 is a perspective view of another embodiment of an integrated electromagnetic navigation and patient positioning device for use in spinal surgery, which provides automated adjustment of the patient positioning device;

FIGS. 7a and 7b are side views of the patient positioning device of FIG. 6 with a modular electromagnetic generator illustrated in various adjusted positions;

FIG. 8 is a perspective view of another patient positioning device with integrated electromagnetic navigation used for knee procedures;

FIGS. 9a-9g illustrate other exemplary patient positioning devices that integrate an electromagnetic navigation system;

FIG. 10 is a perspective view of another patient positioning device having a non-removable integrated electromagnetic navigation system for use during a hip procedure;

FIGS. 11a-11d are additional exemplary embodiments of patient positioning devices having fully integral electromagnetic generators incorporated therein;

FIGS. 12a-12b are other exemplary embodiments of donut or ring-shaped patient positioning devices having either fully integral or removable electromagnetic generators incorporated therein;

FIGS. 13a-13b are other exemplary embodiments of a horseshoe-shaped patient positioning device having either fully integral or removable electromagnetic generators incorporated therein;

FIGS. 14a-14b are additional exemplary embodiments of adjustable patient positioning devices having multiple electromagnetic generators incorporated therein;

FIG. 15 is an exemplary embodiment of an adjustable patient positioning device having the patient retaining devices incorporating electromagnetic generators;

FIG. 16 is another exemplary embodiment of a patient positioning device having removable wedge portions for patient adjustment;

FIG. 17 is an additional exemplary embodiment of a patient positioning device used to retain and support a leg of a patient;

FIG. 18 is another exemplary embodiment of a patient positioning device used for retaining and supporting a leg of a patient; and

FIG. 19 is another exemplary embodiment of a patient positioning device used for retaining and supporting a leg of a patient.

### DETAILED DESCRIPTION OF THE PREFERRED EMBODIMENTS

The following description of the preferred embodiment(s) is merely exemplary in nature and is in no way intended to limit the invention, its application, or uses. Moreover, while the present invention is discussed in detail below with regard to specific types of medical and surgical procedures, the integrated electromagnetic navigation and patient positioning device may be used in any type of medical procedure, including orthopedic, cardiovascular, neurovascular, spinal, soft tissue procedures, lead placement, pain management, radiology procedures, spinal cord stimulations, or any other medical procedures. Moreover, while the patient positioning devices illustrated are typically separate devices, atop an OR table, the patient positioning devices may also be integrated, attached, mounted, or formed directly into an OR table.

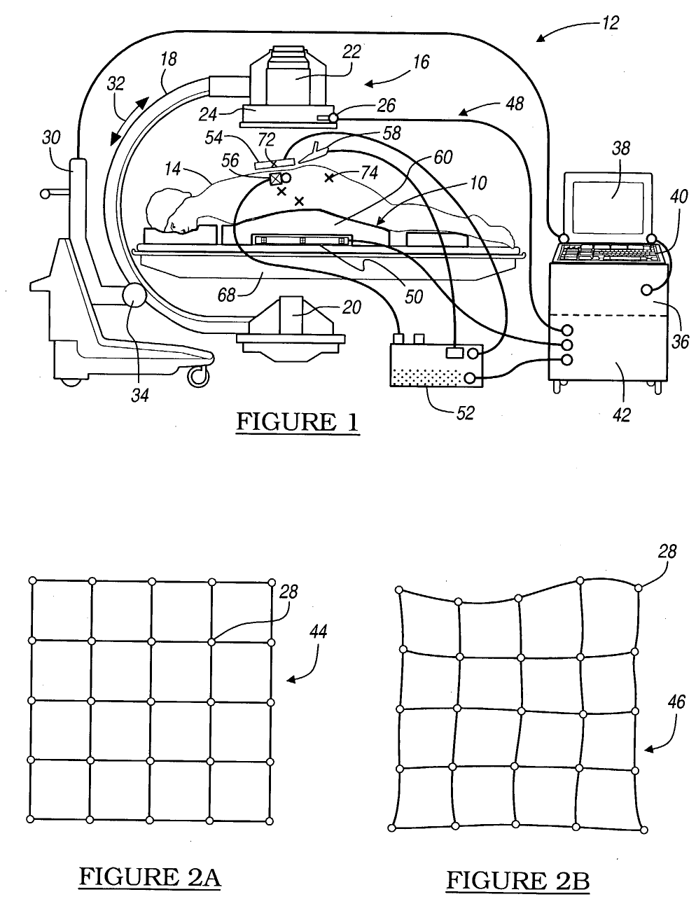

FIG. 1 is a diagram illustrating an exemplary integrated electromagnetic (EM) navigation and patient positioning device 10 employed with an image guided navigation system 12 for use in navigating a surgical instrument or implant during a medical procedure. It should also be noted that the integrated patient positioning device 10 of the present invention may also be used or employed in an image-less based navigation system, further discussed herein. The navigation system 12 may be used to navigate any type of instrument or delivery system, such as a reamer, impactor, cutting block, saw blade, drill guide, drill, robotic arm, catheter, guide wires, needles, drug delivery systems, and cell delivery systems. The navigation system 12 may also be used to navigate any type of implant including orthopedic implants, spinal implants, cardiovascular implants, neurovascular implants, soft tissue implants, or any other devices implanted in a patient 14. The navigation system 12 may also be used to navigate implants or devices that are formed as an assembly or from multiple components where the location and orientation of each component is dependent upon one another to be effective in its use. For example, during a spinal procedure, the display may be used to track and align a spinal screw with a spinal rod to insure attachment of each device.

The navigation system 12 includes an imaging device 16 that is used to acquire pre-operative, real-time, or interoperative images of the patient 14. The imaging device 16 is a fluoroscopic C-arm x-ray imaging device that includes a C-arm 18, an x-ray source 20, an x-ray receiving section 22, an optional calibration and tracking target 24 and optional radiation sensors 26. The optional calibration and tracking target 24 includes calibration markers 28 (see FIGS. 2a-2b), further discussed herein. A C-arm controller 30 captures the x-ray images received at the receiving section 22 and stores the images for later use. The C-arm controller 30 may also control the rotation of the C-arm 18. For example, the C-arm 18 may move in the direction of arrow 32 or rotate about the long axis of the patient 14, allowing anterior or lateral views of the patient 14 to be imaged. Each of these movements involve rotation about a mechanical axis 34 of the C-arm 18. In this example, the long axis of the patient 14 is substantially in line with the mechanical axis 34 of the C-arm 18. This enables the C-arm 18 to be rotated relative to the patient 14, allowing images of the patient 14 to be taken from multiple directions or about multiple planes. An example of a fluoroscopic C-arm x-ray imaging device 16 is the “Series 9600 Mobile Digital Imaging System,” from OEC Medical Systems, Inc., of Salt Lake City, Utah. Other exemplary fluoroscopes include bi-plane fluoroscopic systems, ceiling fluoroscopic systems, cath-lab fluoroscopic systems, fixed C-arm fluoroscopic systems, etc.

In operation, the imaging device 16 generates x-rays from the x-ray source 20 that propagate through the patient 14 and optional calibration and/or tracking target 24, into the x-ray receiving section 22. The receiving section 22 generates an image representing the intensities of the received x-rays. Typically, the receiving section 22 includes an image intensifier that first converts the x-rays to visible light and a charge coupled device (CCD) video camera that converts the visible light into digital images. Receiving section 22 may also be a digital device that converts x-rays directly to digital images, thus potentially avoiding distortion introduced by first converting to visible light. With this type of digital C-arm, which is generally a flat panel device, the calibration and/or tracking target 24 and the calibration process discussed below may be eliminated. Also, the calibration process may be eliminated for different types of medical procedures. Alternatively, the imaging device 16 may only take a single image with the calibration and tracking target 24 in place. Thereafter, the calibration and tracking target 24 may be removed from the line-of-sight of the imaging device 16.

Two dimensional fluoroscopic images taken by the imaging device 16 are captured and stored in the C-arm controller 30. These images are forwarded from the C-arm controller 30 to a controller or work station 36 having a display 38 that may either include a single display 38 or a dual display 38 and a user interface 40. The work station 36 provides facilities for displaying on the display 38, saving, digitally manipulating, or printing a hard copy of the received images. The user interface 38, which may be a keyboard, joy stick, mouse, touch pen, touch screen or other suitable device allows a physician or user to provide inputs to control the imaging device 16, via the C-arm controller 30, or adjust the display settings. The work station 36 may also direct the C-arm controller 30 to adjust the rotational axis 34 of the C-arm 18 to obtain various two-dimensional images along different planes in order to generate representative two-dimensional and three-dimensional images. When the x-ray source 20 generates the x-rays that propagate to the x-ray receiving section 22, the radiation sensors 26 sense the presence of radiation, which is forwarded to the C-arm controller 30, to identify whether or not the imaging device 16 is actively imaging. This information is also transmitted to a coil array controller 42, further discussed herein. Alternatively, a person or physician may manually indicate when the imaging device 16 is actively imaging or this function can be built into the x-ray source 20, x-ray receiving section 22, or the control computer 30.

Fluoroscopic C-arm imaging devices 16 that do not include a digital receiving section 22 generally require the calibration and/or tracking target 24. This is because the raw images generated by the receiving section 22 tend to suffer from undesirable distortion caused by a number of factors, including inherent image distortion in the image intensifier and external electromagnetic fields. An empty undistorted or ideal image and an empty distorted image are shown in FIGS. 2a and 2b, respectively. The checkerboard shape, shown in FIG. 2a, represents the ideal image 44 of the checkerboard arranged calibration markers 28. The image taken by the receiving section 22, however, can suffer from distortion, as illustrated by the distorted calibration marker image 46, shown in FIG. 2b.

Intrinsic calibration, which is the process of correcting image distortion in a received image and establishing the projective transformation for that image, involves placing the calibration markers 28 in the path of the x-ray, where the calibration markers 28 are opaque or semi-opaque to the x-rays. The calibration markers 28 are rigidly arranged in pre-determined patterns in one or more planes in the path of the x-rays and are visible in the recorded images. Because the true relative position of the calibration markers 28 in the recorded images are known, the C-arm controller 30 or the work station or computer 36 is able to calculate an amount of distortion at each pixel in the image (where a pixel is a single point in the image). Accordingly, the computer or work station 36 can digitally compensate for the distortion in the image and generate a distortion-free or at least a distortion improved image 44 (see FIG. 2a). A more detailed explanation of exemplary methods for performing intrinsic calibration are described in the references: B. Schuele, et al., “Correction of Image Intensifier Distortion for Three-Dimensional Reconstruction,” presented at SPIE Medical Imaging, San Diego, Calif., 1995; G. Champleboux, et al., “Accurate Calibration of Cameras and Range Imaging Sensors: the NPBS Method,” Proceedings of the IEEE International Conference on Robotics and Automation, Nice, France, May, 1992; and U.S. Pat. No. 6,118,845, entitled “System And Methods For The Reduction And Elimination Of Image Artifacts In The Calibration Of X-Ray Imagers,” issued Sep. 12, 2000, the contents of which are each hereby incorporated by reference.

While the fluoroscopic C-arm imaging device 16 is shown in FIG. 1, any other alternative imaging modality may also be used or an image-less based application may also be employed, as further discussed herein. For example, isocentric fluoroscopy, bi-plane fluoroscopy, ultrasound, computed tomography (CT), multi-slice computed tomography (MSCT), magnetic resonance imaging (MRI), high frequency ultrasound (HIFU), optical coherence tomography (OCT), intra-vascular ultrasound (IVUS), 2D, 3D or 4D ultrasound, or intraoperative CT or MRI may also be used to acquire pre-operative, real-time or interoperative images or image data of the patient 14. Image datasets from hybrid modalities, such as positron emission tomography (PET) combined with CT, or single photon emission computer tomography (SPECT) combined with CT, could also provide functional image data superimposed onto anatomical data to be used to confidently reach target sights within the areas of interest. It should further be noted that the fluoroscopic C-arm imaging device 16, as shown in FIG. 1, provides a virtual bi-plane image using a single-head C-arm fluoroscope 16 by simply rotating the C-arm 18 about at least two planes, which could be orthogonal planes to generate two-dimensional images that can be converted to three-dimensional volumetric images that can be displayed on the display 38.

The navigation system 12 further includes an electromagnetic navigation or tracking system 48 that includes a transmitter coil array 50, the coil array controller 42, a navigation probe interface 52, an instrument 54 having an electromagnetic tracker, a dynamic reference frame 56 and a pointer probe 58. It should further be noted that the entire tracking system 48 or parts of the tracking system 50 may be incorporated into the imaging device 16, including the work station 36 and radiation sensors 26. Incorporating the tracking system 48 will provide an integrated imaging and tracking system. Any combination of these components may also be incorporated into the imaging system 16, which again can include a fluoroscopic C-arm imaging device or any other appropriate imaging device. Obviously, if an image-less procedure is performed, the navigation and tracking system 48 will be a stand alone unit.

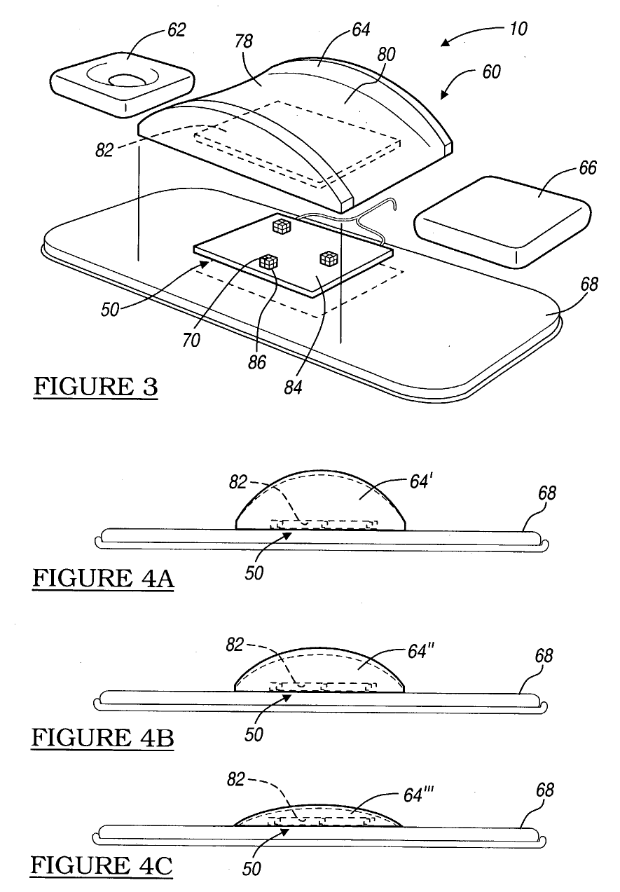

The transmitter coil array 50 is shown incorporated into a patient positioning device 60, further discussed herein. Briefly, the patient positioning device 60, as shown in FIG. 3, includes a head support portion 62, a body support portion 64 and a leg support portion 66 in order to position the patient 14 prone on the operating table 68. The transmitter coil array 50 includes a plurality of coils 70 that are each operable to generate distinct electromagnetic fields into the navigation region of the patient 14, which is sometimes referred to as patient space. Representative electromagnetic systems are set forth in U.S. Pat. No. 5,913,820, entitled “Position Location System,” issued Jun. 22, 1999; U.S. Pat. No. 5,592,939, entitled “Method and System for Navigating a Catheter Probe,” issued Jan. 14, 1997; U.S. Ser. No. 09/698,896, filed Oct. 27, 2000, entitled “Coil Structures and Methods for Generating Magnetic Fields;” U.S. Pat. No. 6,493,573, entitled “Method And System For Navigating A Catheter Probe In The Presence Of Field-influencing Objects,” issued Dec. 10, 2002; and U.S. Pat. No. 5,755,725, entitled “Computer-Assisted Microsurgery Methods And Equipment,” issued May 26, 1998, each of which are hereby incorporated by reference.

The transmitter coil array 50 is controlled or driven by the coil array controller 42. The coil array controller 42 drives each coil 70 in the transmitter coil array 50 in a time division multiplex or a frequency division multiplex manner. In this regard, each coil 70 may be driven separately at a distinct time or all of the coils 70 may be driven simultaneously with each being driven by a different frequency. Upon driving the coils 70 in the transmitter coil array 50 with the coil array controller 42, electromagnetic fields are generated within the patient 14 in the area where the medical procedure is being performed, which is again sometimes referred to as patient space. The electromagnetic fields generated in the patient space induces currents in sensors 72 positioned in the instrument 54, further discussed herein. These induced signals from the instrument 54 are delivered to the navigation probe interface 52 and subsequently forwarded to the coil array controller 42. The navigation probe interface 52 provides all the necessary electrical isolation for the navigation system 12. The navigation probe interface 52 also includes amplifiers, filters and buffers required to directly interface with the sensors 72 in instrument 54. Alternatively, the instrument 54 may employ a wireless communications channel as opposed to being coupled directly to the navigation probe interface 52.

The instrument 54 is equipped with at least one, and may include multiple localization sensors 72. In this regard, the instrument 54 may include an orthogonal pair coil sensor 72, a tri-axial coil sensor 72, multiple or single coil sensors 72 positioned about the instrument 54. Here again, the instrument 54 may be any type of medical instrument or implant. For example, the instrument 54 may be a catheter that can be used to deploy a medical lead, capture a biopsy to be used for tissue ablation, or be used to deliver a pharmaceutical agent. The instrument 54 may also be an orthopedic instrument, used for an orthopedic procedure, such as reamers, impactors, cutting blocks, saw blades, drills, etc. The instrument 54 may also be any type of neurovascular instrument, cardiovascular instrument, soft tissue instrument, etc. Finally, the instrument 54 may be an implant that is tracked, as well as any other type of device positioned and located within the patient 14. These implants can include orthopedic implants, neurovascular implants, cardiovascular implants, soft tissue implants, or any other devices that are implanted into the patient 14. Particularly, implants that are formed from multiple components where the location and orientation of each component is dependent upon the location and orientation of the other component.

In an alternate embodiment, the electromagnetic sources or generators may be located within the instrument 54 and one or more receiver coils may be provided externally to the patient 14 forming a receiver coil array similar to the transmitter coil array 50. In this regard, the sensor coils 72 would generate electromagnetic fields, which would be received by the receiving coils in the receiving coil array similar to the transmitter coil array 50. Other types of localization or tracking may also be used with other types of navigation systems, which may include an emitter, which emits energy, and a receiver that detects the energy at a position away from the emitter. This change in energy, from the emitter to the receiver, is used to determine the location of the receiver relative to the emitter. These types of localization systems can include ultrasound, sonic, electromagnetic, hybrid systems, etc. An additional representative alternative localization and tracking system is set forth in U.S. Pat. No. 6,235,038, entitled “System For Translation Of Electromagnetic And Optical Localization Systems,” issued May 22, 2001. Alternatively, the localization system may be a hybrid system that includes components from various systems. With each of these types of systems, the relevant equipment would be integrated and incorporated into the patient positioning device 60.

The dynamic reference frame 56 of the electromagnetic tracking system 48 is also coupled to the navigation probe interface 52 to forward the information to the coil array controller 42. The dynamic reference frame 56 is a small magnetic field detector or any other type of detector/transmitter that is designed to be fixed to the patient 14 adjacent to the region being navigated so that any movement of the patient 14 is detected as relative motion between the transmitter coil array 50 and the dynamic reference frame 56. This relative motion is forwarded to the coil array controller 42, which updates registration correlation and maintains accurate navigation, further discussed herein. The dynamic reference frame 56 can be configured as a pair of orthogonally oriented coils, each having the same center or may be configured in any other non-coaxial coil configuration. The dynamic reference frame 56 may be affixed externally internally, percutaneously, subcutaneous and minimally invasive to the patient 14, adjacent to the region of navigation, such as the patient's spinal region, as shown in FIG. 1 or on any other region of the patient. The dynamic reference frame 56 can be affixed to the patient's skin, by way of a stick-on adhesive patch. The dynamic reference frame 56 may also be removably attachable to fiducial markers 74 also positioned on the patient's body. For example, representative dynamic reference frames and fiducial markers are set forth in U.S. Pat. No. 6,381,485, entitled “Registration Of Human Anatomy Integrated For Electromagnetic Localization” issued Apr. 30, 2002 and U.S. Pat. No. 6,499,488, entitled “Surgical Sensor”, issued Dec. 30, 2002, each of which are hereby incorporated by reference.

Alternatively, the dynamic reference frame 56 may be internally attached, for example, to the spine or vertebral bodies, pelvis or femur of the patient using bone screws that are attached directly to the bone. This provides increased accuracy since this will track any motion of the bone. Moreover, multiple dynamic reference frames 56 may also be employed to track the position of one, two or more bones relative to a joint. For example, one dynamic reference frame 56 may be attached to one vertebra, while a second dynamic reference frame 56 may be attached to a second vertebra during a spinal procedure. In this way, motion of the spine may be detected by the dual dynamic reference frames 56.

Briefly, the navigation system 12 operates as follows. The navigation system 12 creates a translation map between all points in the radiological image generated from the imaging device 16 and the corresponding points in the patient's anatomy in patient space. After this map is established, whenever a tracked instrument 54 is used, the work station 36 in combination with the coil array controller 42 and the C-arm controller 30 uses the translation map to identify the corresponding point on the pre-acquired image, which is displayed on display 38. This identification is known as navigation or localization. An icon representing the localized point or instrument is shown on the display 38.

To enable navigation, the navigation system 12 must be able to detect both the position of the patient's anatomy 14 and the position of the surgical instrument 54. Knowing the location of these two items allows the navigation system 12 to compute and display the position of the instrument 54 in relation to the patient 14. The tracking system 48 is employed to track the instrument 54 and the anatomy simultaneously. While the display 38 is configured to show the instrument.

The tracking system 48 essentially works by positioning the transmitter coil array 50 adjacent to the patient space to generate a low-energy magnetic field generally referred to as a navigation field. Because every point in the navigation field or patient space is associated with a unique field strength, the electromagnetic tracking system 48 can determine the position of the instrument 54 by measuring the field strength at the sensor 72 location. The dynamic reference frame 56 is fixed to the patient 14 to identify the location of the patient 14 in the navigation field. The electromagnetic tracking system 48 continuously recomputes the relative position of the dynamic reference frame 56 and the instrument 54 during localization and relates this spatial information to patient registration data to enable image guidance of the instrument 54 within the patient 14.

Patient registration is the process of determining how to correlate the position of the instrument 54 on the patient 14 to the position on the diagnostic, pre-acquired, or real-time images. To register the patient 14, the physician or user will select and store particular points from the pre-acquired images and then touch the corresponding points on the patient's anatomy with a pointer probe 58. The navigation system 12 analyzes the relationship between the two sets of points that are selected and computes a match, which correlates every point in the image data with its corresponding point on the patient's anatomy or the patient space. The points that are selected to perform registration are the fiducial arrays or landmarks 74. Again, the landmarks or fiducial points 74 are identifiable on the images and identifiable and accessible on the patient 14. The landmarks 74 can be artificial landmarks 74 that are positioned on the patient 14 or anatomical landmarks 74 that can be easily identified in the image data. The system 12 may also perform 2D to 3D registration by utilizing the acquired 2D images to register 3D volume images by use of contour algorithms, point algorithms or density comparison algorithms, as is known in the art.

In order to maintain registration accuracy, the navigation system 12 continuously tracks the position of the patient 14 during registration and navigation. This is necessary because the patient 14, dynamic reference frame 56, and transmitter coil array 50 may all move during the procedure, even when this movement is not desired. Therefore, if the navigation system 12 did not track the position of the patient 14 or area of the anatomy, any patient movement after image acquisition would result in inaccurate navigation within that image. The dynamic reference frame 56 allows the electromagnetic tracking device 48 to register and track the anatomy. Because the dynamic reference frame 56 is rigidly fixed to the patient 14, any movement of the anatomy or the transmitter coil array 50 is detected as the relative motion between the transmitter coil array 50 and the dynamic reference frame 56. This relative motion is communicated to the coil array controller 42, via the navigation probe interface 52, which updates the registration correlation to thereby maintain accurate navigation. This type of monitoring is particularly relevant when the patient 14 is moved to different shaped patient positioning devices or when the patient positioning device is adjusted, further discussed herein.

It should also be understood that localization and registration data may be specific to multiple targets. For example, should a spinal procedure be conducted, each vertebra may be independently tracked and the corresponding image registered to each vertebra. In other words, each vertebra would have its own translation map between all points in the radiological image and the corresponding points in the patient's anatomy in patient space in order to provide a coordinate system for each vertebra being tracked. The tracking system 48 would track any motion in each vertebra by use of a tracking sensor 72 associated with each vertebra. In this way, dual displays 38 may be utilized, where each display tracks a corresponding vertebra using its corresponding translation map and a surgical implant or instrument 54 may be registered to each vertebra and displayed on the display 38 further assisting an alignment of an implant relative to two articulating or movable bones. Moreover, each separate display in the dual display 38 may superimpose the other vertebra so that it is positioned adjacent to the tracked vertebra thereby adding a further level of information.

As an alternative to using the imaging system 16, in combination with the navigation and tracking system 48, integrated patient positioning device 10 can be used in an imageless manner without the imaging system 16. In this regard, the navigation and tracking system 48 may only be employed and the probe 62 may be used to contact or engage various landmarks on the patient. These landmarks can be bony landmarks on the patient, such that upon contacting a number of landmarks for each bone, the workstation 36 can generate a three-dimensional model of the bones. This model is generated based upon the contacts and/or use of atlas maps. The workstation 36 may also generate a center axis of rotation for the joint or planes, based upon the probe contacts. Alternatively, the tracking sensor 72 may be placed on the patient's anatomy and the anatomy moved and correspondingly tracked by the tracking system 48. For example, placing a tracking sensor 72 on the femur and fixing the pelvis in place of a patient and rotating the leg while it is tracked with the tracking system 48 enables the work station 36 to generate a center of axis of the hip joint by use of kinematics and motion analysis algorithms, as is known in the art. If the pelvis is not fixed, another tracking sensor 72 may be placed on the pelvis to identify the center of axis of the hip joint.

If a tracking sensor 72 is placed on the femur and a tracking sensor 72 is placed on the tibia, upon moving this portion of the anatomy, a center of axis of the knee joint may be identified. Likewise, by placing a separate tracking sensor 72 on two adjacent vertebra and articulating the spine, the center of axis of the spinal region can also be identified. In this way, a model based on the center of the particular joint may be designated and identified using known kinematics and/or motion analysis algorithms or atlas maps or tables, as is known in the art. Movement of the instrument or implant 54 may then be tracked in relation to this model to properly align the instrument or implant 54 relative to the model, which may be a two-dimensional or three-dimensional model.

Turning now to FIG. 3, the integrated patient positioning device 10 will be discussed in further detail. Here again, the integrated patient positioning device 10 includes the patient positioning device or three-dimensional contoured support 60 comprising the head support 62, the body support 64 and the leg support 66. These positioning devices may be formed from rigid frames enclosed with a foam material or may be simply a support foam material. The material used can be carbon fiber, ceramic, laminates, poly based, etc. Alternatively, these devices may be formed of any structure or material to support the patient 14. The patient positioning device 60 may also be radiolucent and electromagnetically compatible. In this regard, the patient positioning devices may be substantially invisible to a fluoroscopic image on the imaging device 16 and may also not interfere with the transmitter coil array 50. In this regard, there may be little shielding or interference effect with the patient positioning device 60 in relation to the coil array 50, thereby reducing any shielding or distortion of an electromagnetic field generated by the coil array 50. Alternatively, the tracking system 48 may initially be calibrated or take into consideration any field influencing effects of the patient positioning device 60 in relation to the coil array 50. For example, a system that takes into consideration distortions, is set forth in U.S. Pat. No. 6,493,573, entitled “Method And System For Navigating A Catheter Probe In The Presence Of Field-Influencing Objects”, issued Dec. 10, 2002, the contents of which are hereby incorporated by reference.

The patient positioning device 60, shown in FIG. 3 is positioned atop the operating table 68 so that the patient 14 is positioned prone to the operating table 68. In other words, the patient positioning device or support 64 includes a three-dimensional arcuate contoured surface 78 in order to position the desired surgical site at the apex 80 of the support 64. Obviously, the patient positioning device 60 will also support the patient's weight and provide improved access to the anatomy of the patient 14. Here again, the patient positioning device 60 may be incorporated directly into the OR table 68.

The center of the patient positioning device 64 defines a cutout region 82 that is operable to nestingly receive the coil array 50, which is placed atop the OR table 68. Alternatively, the OR table 68 may define a recess, which nestingly receives the coil array 50 and can therefore act alone as the patient positioning device. The coil array 50 is generally formed within a rectangular housing 84, which supports three tracking cubes 86. Alternatively, the housing 84 may be eliminated and the patient positioning device 64 may simply define three smaller recesses to receive three individual cubes 86. Each cube 86 generally include at least three orthogonally or any other oriented coils 70, such as that disclosed in detail in U.S. Pat. No. 5,913,820, entitled “Position Location Sensor,” issued Jun. 22, 1999, which is hereby incorporated by reference. Alternatively, the housing 84 may contain the coils 70 configured in any other configuration, for example, the configuration, set out in U.S. Pat. No. 5,592,939, entitled “Method And System For Navigating A Catheter Probe,” issued Jan. 14, 1997, which is hereby incorporated by reference. Alternatively, the coils may be positioned in any other configuration, which is able to provide or create a working volume or patient space large enough in which the surgical instrument 54 can be navigated. By providing the recess 82, which nestingly receives the coil array 50, a standard surgical arena is maintained, while still providing navigation of the surgical instrument 54. In this way, a convenient and economical mechanism to position the coil array controller 50 in an inobtrusive way to enable the surgeon to perform the medical procedure unobstructed and unencumbered is achieved.

It should be pointed out that while the coil array 50, is shown positioned atop the OR table 68 and in the bottom of the patient positioning device 64 within recess 82, the coil array 50 may be located anywhere within the patient positioning device 60. In other words, a horizontal recess may be formed in the patient positioning device 60 to slidably receive the coil array 50. Alternatively, vertical or angular recesses may also be provided to receive the coil array 50. Still further, the coil array 50 does not need to be positioned directly atop the OR table, but can be elevated above the OR table by being mounted within the patient positioning device 60.

Referring now to FIGS. 4a-4c, different size patient positioning devices 64′, 64′, and 64′″ are illustrated. In this regard, each arcuately shaped patient positioning device is shown having a different angle of elevation and incline. Additionally, each of the arcuate patient positioning devices includes the same size recess 82 to receive the coil array 50. In this way, a surgeon can simply select the appropriate size patient positioning device 64′, 64″ or 64′″ depending on the particular spinal procedure being performed and on the patient's shape and size, without the need for changing the coil array 50. Thus, by using a universal coil array 50 having the same size housing 84, any size or configuration of a patient positioning device can be made to accommodate this coil array 50 by simply having the same size recess 82. This again provides a cost-effective and simple way for enabling different shaped patient positioning devices to be used with one common coil array 50. Here again, the coil array 50 may have a rectangular-shaped housing 84, a circular housing, a triangular housing, U-shaped housing or any other shaped housing or may simply have three separate recesses in order to accommodate the three tracking cubes 86 or a single tracking cube 86. It should also be pointed out that any number of tracking cubes 86 may be used or any number of coils within the coil array 50 may be used and oriented in any orientation with or without the use of the tracking cubes. In this regard, the coils may be overlapping coils, spiral wound coils, planar configured coils, etc.

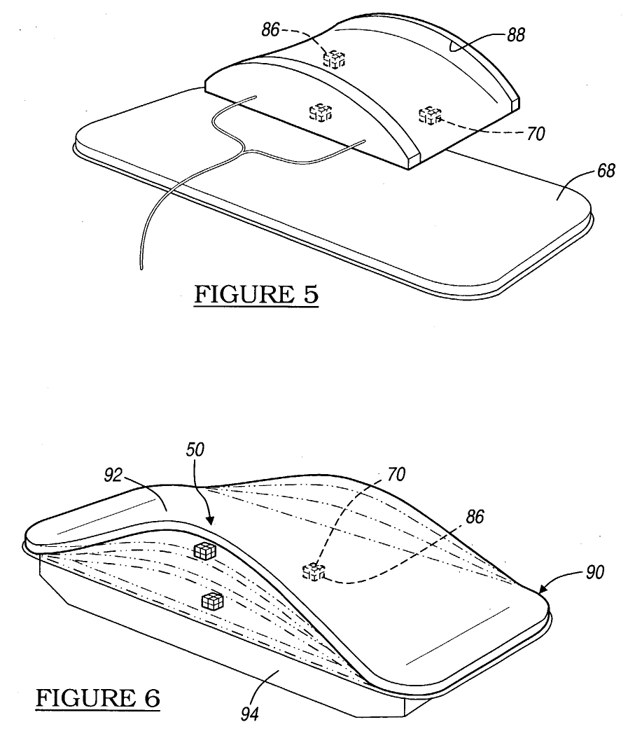

FIG. 5 illustrates another arcuate patient positioning device 88 having substantially the same shape and contour as the patient positioning device 64. The only difference is that each set of coils 70, either formed on the tracking cube 86 or formed in other ways, are formed integrally and incorporated directly within the patient positioning device 88. In this way, in order to perform a medical procedure that is electromagnetically navigated, the patient positioning device 88 simply needs to be placed atop the OR table 68. Here again, the coil array 50 may be incorporated directly into any type of patient positioning device having any type of configuration for any type of medical procedure, further discussed herein. By incorporated the coil array 50 directly and non-removably into the patient positioning device, no assembly or stacking of the patient positioning device over the coil array 50 is necessary. However, should different sized patient positioning devices 88 be necessary, as shown in FIGS. 3a-3c, three coil arrays 50 would be required with one being incorporated directly into each patient positioning device 88 in a non-removable manner.

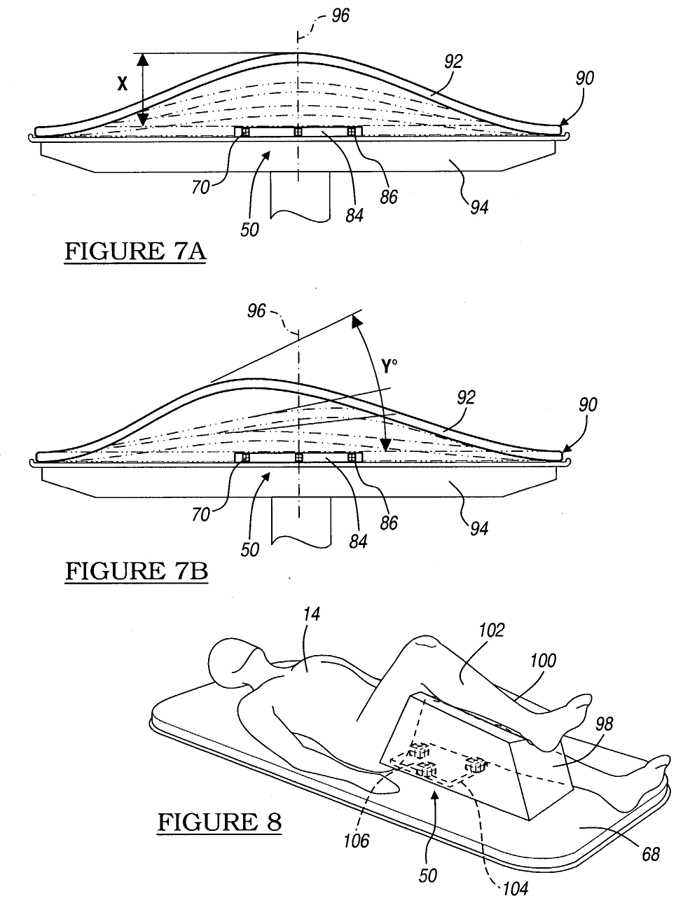

Turning to FIGS. 6, 7a and 7b, an automated adjustable OR table or patient positioning device 90 is shown. The OR table 90, shown in FIG. 6, includes the coil array 50 incorporated directly or integral within the OR table 90, while in FIGS. 7a and 7b, the coil array 50 is a modular coil array positioned within the housing 84 so that the coil array may be removed from the OR table if necessary. The OR table 90 may be an adjustable table, as is known in the art. For example, table 90 may be formed similar to the table identified in U.S. Pat. No. 5,239,716, entitled “Surgical Spinal Positioning Frame”, issued Aug. 31, 1993, which is hereby incorporated by reference. In this regard, the table 90 includes a flexible operating surface 92 and a rigid lower frame 94. The flexible surface 92 enables the height or the arch of the operating surface to be adjusted along a height X, as shown in FIG. 7a. Likewise, the angular orientation of the operating surface can be adjusted relative to an apex 96, as identified by angle Y°, as shown in FIG. 7b. Here again, the incline angle and the decline angle may be adjusted accordingly. Positioned atop the rigid structure 94 is the coil array 50, which is integrally constructed within the OR table 90 in FIG. 6. Each tracking cube 86 having the corresponding coils 70 is fixed relative to the rigid structure 94, so that when the top 92 is adjusted, as is shown in FIG. 7a and 7b, the coils 70 remain fixed at a known location.

The placement of the tracking cubes 86 again provides a sufficiently large patient space or working volume in which to navigate the surgical instrument 54. Here again, the working surface 92 and surrounding mechanical adjustment structure may be constructed of appropriate materials to reduce shielding effects and electromagnetic interference of the electromagnetic field generated by the coil array 50. It should further be noted that the coil array 50 shown in FIGS. 7a and 7b is not integral within the table 90, but is simply slid into an appropriate recess within the table 90 to accommodate the coil array 50. Alternatively, any other shaped housing or the individual tracking cubes may also be simply removably positioned in place within the OR table 90.

Other shaped patient positioning devices may also be utilized, which incorporate either the removable modular coil array 50 or the non-removable integrated coil array 50. For example, the patient positioning device 98, as shown in FIG. 8, is used during knee surgery to position the patient's knee in full flexion. The patient positioning device 98 includes the appropriately shaped groove 100 to cradle and secure the leg 102. The patient positioning device 98 also defines a recess 104, which receives the coil array 50 housed within a smaller rectangular housing 106. As previously indicated, various shaped housings may also be provided to accommodate various sized patient positioning devices. For example, the hospital may have two to three different shape and size housings for the coil array 50 and dozens of patient positioning devices that are capable to receive one of these housings that houses the coil array 50. Alternatively, as was previously discussed, each patient positioning device may include its own integrated coil array 50.

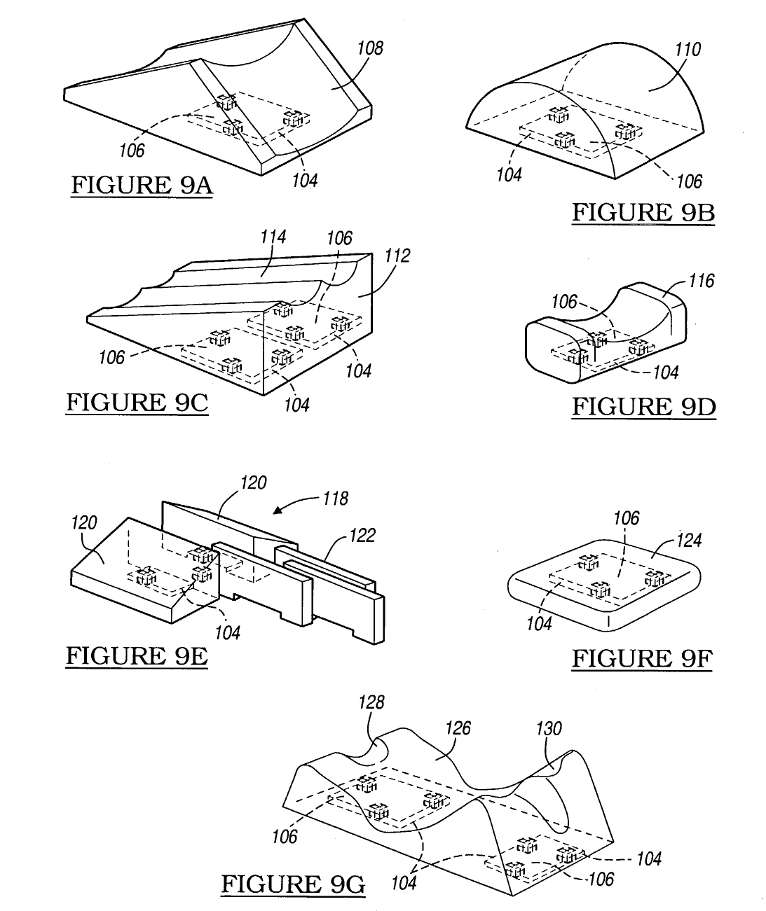

FIGS. 9a-9g illustrate various shaped patient positioning devices used for various types of medical procedures and surgeries. Each of these patient positioning devices is configured to receive a removable modular coil array 50. However, these patient positioning devices may also include an integral or non-removable coil array 50.

FIG. 9a illustrates a wedge-shaped patient positioning device 108, which defines the recess 104 to receive the housing 106 holding the coil array 50. The patient positioning device 108 may be used for spinal procedures, pelvic procedures, or other appropriate procedures.

FIGS. 9b illustrates an arcuate-shaped patient positioning device 110 also defining the shaped recess 104. The patient positioning device 110 may be used for spinal, abdominal or leg surgeries.

FIG. 9c illustrates a triangular patient positioning device 112 that defines two recesses 104 to receive two housings 106, which house two coil arrays 50. The triangular shaped patient positioning device 112 also defines two arcuately shaped grooves 114 to retain both legs of the patient 14. By providing two recesses 104 to receive two housings 106, either two coil arrays 50 may be used when surgery or procedures are being performed on two legs or a single coil array 50 may be utilized depending on which leg is being operated upon.

FIG. 9d illustrates a cup-shaped patient positioning device 116, which defines the smaller recess 104 to receive the smaller housing 106. The patient positioning device 116 may be used to nestingly receive the head of the patient 14 or the waist of the patient 14.

FIG. 9e illustrates a patient positioning assembly 118, which includes several individual patient positioning devices. Patient positioning assembly 118 includes two angled blocks 120 and a plurality of intermediate blocks 122. The blocks 120 and 122 again define the recess 104 to receive the housing 106, which houses the coil array 50. By providing multiple intermediate blocks 122, the patient positioning assembly 108 may be adjusted to accommodate various sized patients 14, which may be laid atop the patient positioning assembly 118. While three intermediate blocks 122 are shown, any number of intermediate blocks may be utilized to support the patient 14.

FIG. 9f illustrates a rectangular-shaped patient positioning device 124 also defining the recess 104. The rectangular-shaped patient positioning device can again be made out of foam to support different areas of the patient, such as a foot, leg, hand, etc.

FIG. 9g illustrates a patient positioning or retaining device 126, which retains the entire patient 14. In this regard, a head retaining groove 128 retains the head, while two grooves 130 align and retain the legs of the patient 14. The patient positioning device 126 defines two recesses 104 to receive the housing 106 containing the coil array 50. In this regard, should the chest area of the patient be the area of interest, the recess 104 in that area will retain the coil array 50. Should the leg or knee area be the area of interest, the recess 104 positioned adjacent the leg area may be utilized. Alternatively, two coil arrays 50 may be utilized to provide a working volume substantially throughout the patient area.

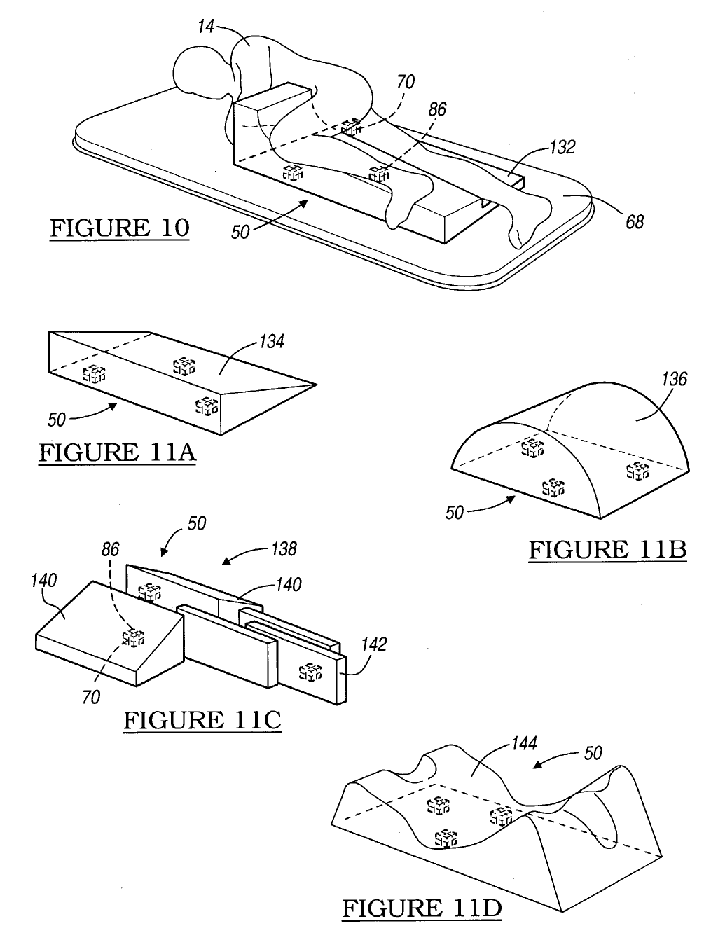

Turning to FIG. 10, an incline patient positioning device 132 is illustrated to position the patient's legs appropriately for hip surgery. The patient positioning device 132 is configured to have the coil 70 positioned about the tracking cubes 86 integrally formed in the patient positioning device 132. Here again, the modular coil array 50 may also be utilized as opposed to integrally formed coils.

Referring to FIGS. 11a-11d, exemplary patient positioning devices having integrally formed coils are illustrated with the understanding that the modular coil array 50 may also be utilized. In this regard, FIG. 11a illustrates a triangular wedge-shaped patient positioning device 134 housing the integral coil array 50. FIG. 11b illustrates an arcuate-shaped patient positioning device 136 integrally housing the coil array 50. FIG. 11c illustrates a patient positioning assembly 138 integrally housing the coil array 50. The end pieces 140 can each contain a tracking cube 86 housing the coils 70 and a center intermediate member 142 may also containing a single tracking cube 86 housing the coils 70. FIG. 11d illustrates a full patient positioning device 144 that integrally houses the coil array 50 in order to provide a working volume throughout the entire patient 14.

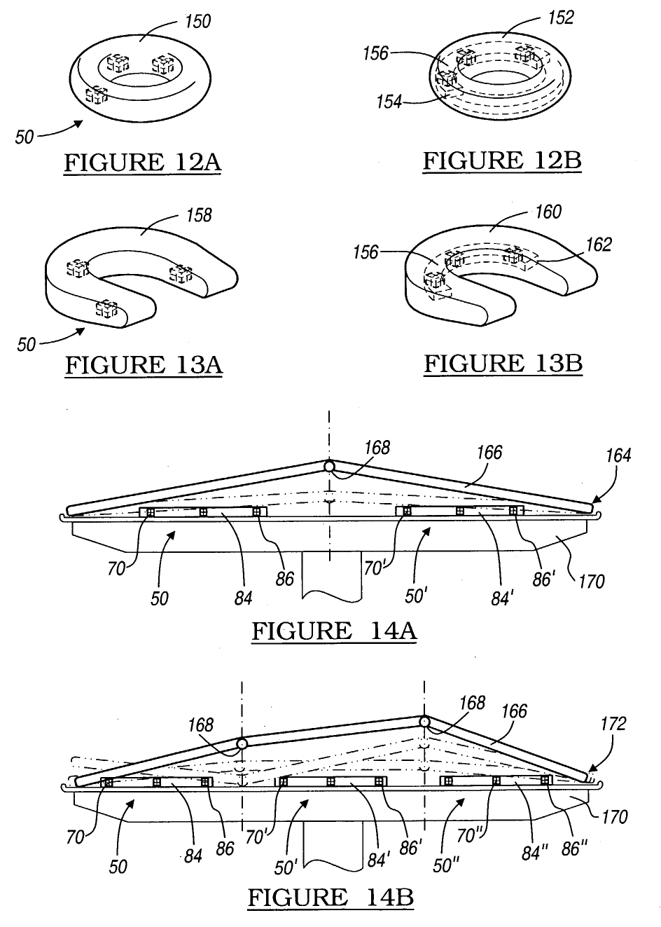

In FIG. 12A, a donut or ring-shaped patient positioning device 150 is shown integrally housing the coil array 50. In FIG. 12B, a patient positioning device 152 is illustrated that defines a circular recess 154 that retains the coil array 50 housed within a U-shaped housing 156. By providing a circular or ring-shaped recess 154, the U-shaped housing 156 that houses the coil array 50 may be rotated substantially about the patient positioning device 152. The patient positioning devices 150 and 152 are generally used to retain the head of a patient during cranial or facial procedures.

Turning to FIG. 13A, a U-shape or a horseshoe-shaped patient positioning device 158 is illustrated integrally housing the coil array 50. In FIG. 13B, another U-shaped or horseshoe-shaped patient positioning device 160 is illustrated, which removably receives the housing 156 within a U-shaped recess 162. Here again, the patient positioning devices 158 and 160 are generally used to retain or support the head of the patient during a cranial or facial procedure.

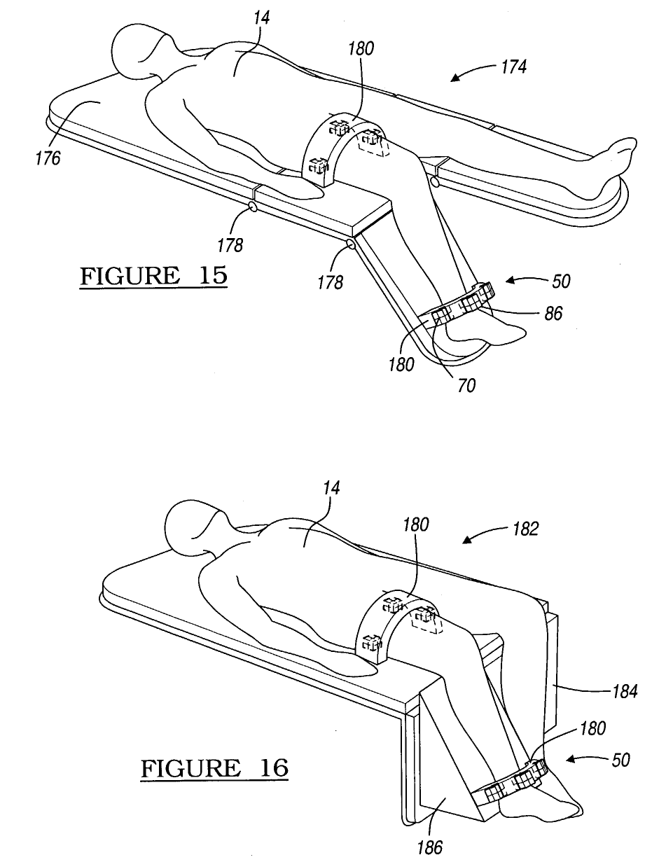

Referring now to FIGS. 14A and 14B, two different embodiments of an adjustable OR table or patient positioning device are shown. In this regard, FIG. 14A illustrates an adjustable OR table 164 having a support surface 166 that is hinged at an apex, via hinge 168. Positioned within the OR table 168 are two coil arrays 50 and 50′, shown separated between the hinge area 168 and housed within housings 84 and 84′. Here again, the OR table 164 includes the hinge top 166 and a support frame 170 that supports both the patient 14 and the pair of coil arrays 50. By providing a separate coil array positioned on each end of the OR table 164, navigation throughout the entire patient 14 may be achieved. Alternatively, the navigation system 48 may select each coil array 50 to utilize depending upon the amount of interference caused by the operating room environment. In this regard, should the coil array 50 located on the left side of the OR table 164 be located in an area of high interference because there any adjacent interfering metal objects, the right coil array 50′ may be utilized to create the navigation space. Moreover, navigation between the coil arrays 50 and 50′ may also be desirable should the interference levels change in the OR environment. For example, this may exist when rotating the C-arm from one location to another, thereby changing potential interfering fields.

The second OR table or patient positioning device 172 is illustrated in FIG. 14B and also includes the operating surface 166 and the frame 170. The OR table 172, however, includes two hinges 168 enabling even further adjustment, such as height and angle. The OR table 172 also includes three coil arrays 50, 50′, and 50′. This again enables the surgeon to select either one coil array or multiple coil arrays to be used during the medical procedure. Selection of the coil arrays can depend upon the amount of interference, the area where the medical procedure is being performed and subsequent changes in the OR environment during the medical procedure. In some applications, the navigation system 48 may auto-select between which coil array to use to navigate. The auto-selectability determination is based on various criteria, such as the area to be navigated, the size of the area to be navigated, interference, etc. The navigation system 48 may also average both navigation spaces from the coil arrays to make these determinations. The coil arrays may also all be driven simultaneously, thereby providing overlapping navigation volumes to navigate larger areas. The navigation system 48 may also switch between coil arrays, depending on where the navigation is occurring so that hand-offs between coil arrays can be achieved, providing a seamless navigated area.

Referring to FIG. 15, OR table or patient positioning device 174 is illustrated for use during knee surgery. In this regard, the OR table 174 supports the patient 14, via patient support platform 176. Patient support platform 176 includes multiple hinges 178 enabling various adjustments of the support surface 176. Upon adjusting the OR table 174 as illustrated, the patient 14 is positioned with the right knee in partial flexion. A pair of U-shaped clamps 180 are used to retain both the ankle and hip area of the patient 14. Housed within each clamp 180 is the coil array 50 that includes the cubes 86, having the wrapped coils 70. Positioning of each coil array 50 adjacent to the flexed knee area enables the fields generated by each coil to converge to provide accurate navigation space around the knee because of the distance between the actual coil array 50 and the navigated knee area. Also, the hinge 178 enables adjustment or flexion of the knee during the medical procedure. By providing two coil arrays 50, both coil arrays 50 may be utilized during the navigation of the knee area or one coil array may be selected, depending upon which coil array has less interference or depending upon changes in the OR environment during the medical procedure.

Referring to FIG. 16, another OR table or patient positioning device 182 is illustrated that includes a hinged portion 184. The patient 14 is again restrained with the pair of U-shaped restraining devices or clamps 180 housing the coil arrays 50. In order to provide for further adjustment regarding the procedure, a wedge shape support 186 is positioned to support the calf area of the patient 14. Should other angles of flexion be desired, the wedge 186 may simply be replaced with a different shaped wedge having a different flexion angle. It should further be noted that while the coil arrays 50 are shown in the U-shaped clamps, these coils arrays may be positioned within the wedge 186, along with the corresponding OR table near the area of navigation desired.

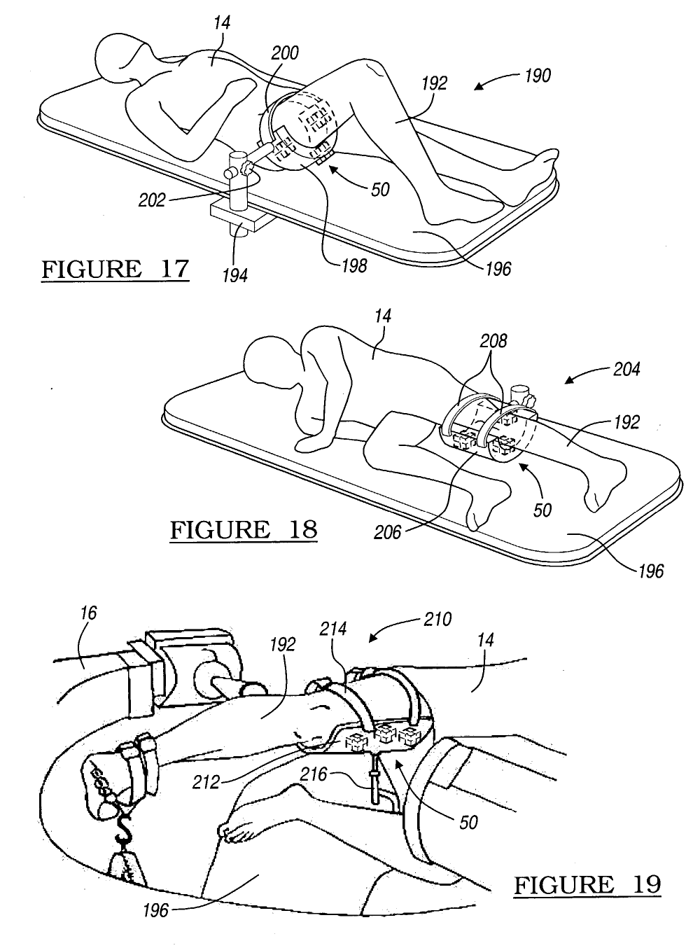

Turning to FIG. 17, another OR table or patient positioning device 190 is illustrated for use in supporting the leg 192 of the patient 14. Patient positioning device 190 includes a bracket 194 that is attached to the side of an OR table 196. The bracket 194 enables the patient positioning device 190 to be slid along the entire length of the OR table 196, depending on the particular procedure utilized. While the bracket 194 is shown attached to the OR table 196, it should be understood that the bracket 194 can be integrated or incorporated directly within the OR table 196. Attached to the bracket 194 is a U-shaped patient positioning bolster or support 198 housing the coil array 50. As shown in FIG. 17, the leg 192 of the patient 14 is supported by the U-shaped support 198 and is retained within the support 198, via an adjustable strap 200. The orientation of the support 198 may be adjusted, via adjustment knob 202, which enables rotational adjustment and also adjustment transversely across the OR table 196. The support 198 may also pivot about bracket 194 and the height may also be adjusted relative to the bracket 194. The patient positioning device 190 supports the leg 192 of the patient 14 for use in different types of surgeries involving legs, such as orthopedic hip replacement, orthopedic knee replacement, or other types of medical procedures.

Referring to FIG. 18, a patient positioning device 204 is illustrated in association with the OR table 196. The patient positioning device 204 is substantially similar to the patient positioning device 190, except that it includes a wider U-shaped support or bolter 206 and a pair of straps 208 to retain the leg 192 of the patient 14. Again, the patient positioning support 206 houses the coil array 50. While the coil array 50 is shown in FIGS. 17 and 18 as being integral within the corresponding supports, again the coil arrays 50 may be housed in other shaped housings to be removably incorporated into the corresponding supports. Again, the patient positioning device 204 restrains the leg 192 of the patient 14 to enable various types of surgeries in this area to be performed accurately.

Referring to FIG. 19, a patient positioning device 210 is illustrated in association with the OR table 196. The patient positioning device 210 is substantially similar to the patient positioning device 204, except that it is removably mounted to an end of the OR table 196. The patient positioning device 210 again includes a U-shaped support 212 and a pair of straps 214 to retain the leg 192 of the patient 14. The U-shaped support 212 again houses the coil array 50. Illustrated adjacent to the patient positioning device 210 is the fluoroscopic C-arm x-ray imaging device 16. The patient positioning device 210 is movably attached to the OR table 196, via an adjustable bracket 216 that is slid into a conventional slot within the OR table 196. The bracket 216 may be adjusted transversly along either end of the OR table 16, can be pivoted about its axis, as well as provide height adjustment, relative to the top of the OR table 196. Again, the patient positioning device 210 supports the leg 192 for use in different types of medical procedures involving this area of interest.

The integrated electromagnetic navigation and patient positioning devices disclosed herein provide a cost effective three-dimensional contoured patient support to integrate either a removable modular or non-removable coil array 50 into the surgical environment and without obstructing the surgeon performing the procedure. The patient positioning devices disclosed are simply exemplary shapes of positioning devices for use with various types of medical procedures. Other representative types of patient positioning devices, are set forth in U.S. Pat. No. 6,182,663, entitled “Patient Positioning Apparatus and Method for Spinal Tap, issued Feb. 6, 2001; U.S. Pat. No. 4,908,892, entitled, “Spinal Surgery Chest Bolster”, issued Mar. 20, 1990; U.S. Pat. No. 5,239,716, entitled “Surgical Spinal Positioning Frame”, issued Aug. 31, 1993; and U.S. Pat. No. 6,123,680, entitled “Centrifugal Force Device And Method For Treatment Of Orthopedic Spinal Disorders”, issued Sep. 26, 2000; which are each hereby incorporated by reference. Each patient positioning device either includes a removable modular coil array assembly or the coil array assembly can be integrally and non-removably formed within the patient positioning device. The patient positioning devices can also define standard sized recesses to accept standard sized and shaped coil arrays. Moreover, while three electromagnetic tracking cubes 86 are illustrated having three orthogonal coils 70, these configurations are merely exemplary and any other coil configuration may be utilized, which either includes tracking cubes or includes simply a housing containing oriented coils.

The description of the invention is merely exemplary in nature and, thus, variations that do not depart from the gist of the invention are intended to be within the scope of the invention. Such variations are not to be regarded as a departure from the spirit and scope of the invention.

What is claimed is:

1. A patient positioning device to position a patient during a navigated medical procedure, said patient positioning device comprising:
a contoured patient support operable to position the patient in a desired manner; and
a portion of a navigation system integrated within said patient support, wherein the navigated medical procedure may be performed in a substantially unobstructed manner.

2. The patient positioning device as defined in claim 1 wherein said navigation system is an electromagnetic navigation system.

3. The patient positioning device as defined in claim 2 wherein said portion of said electromagnetic navigation system is a coil array.

4. The patient positioning device as defined in claim 3 wherein said coil array is a transmitting coil array operable to generate an electromagnetic field in a patient space.

5. The patent positioning device as defined in claim 3 wherein said coil array is a receiving coil array operable to receive an electromagnetic field.

6. The patient positioning device as defined in claim 3 wherein said coil array is removably positioned within said contoured patient support.

7. The patient positioning device as defined in claim 6 wherein said removable coil array is configured within a housing operable to be removably positioned within said contoured patient support.

8. The patient positioning device as defined in claim 6 wherein said contoured patient support defines a recess operable to removably receive said removable coil array.

9. The patient positioning device as defined in claim 8 further comprising a second contoured patient support operable to position the patient in a second desired manner, said second contoured patient support defining a second recess where said second recess is substantially the same size as said recess in said contoured patient support and operable to removably receive said removable coil array.

10. The patient positioning device as defined in claim 1 wherein said contoured patient support is positioned atop an operating room table.

11. The patient positioning device as defined in claim 1 wherein said contoured patient support is formed integral with an operating table.

12. The patient positioning device as defined in claim 1 wherein said contoured patient support is attached to an operating table.

13. The patient positioning device as defined in claim 1 wherein said contoured patient support is adjustable.

14. The patient positioning device as defined in claim 1 wherein said contoured patient support is operable to position a patient in a desired manner to perform a surgery selected from a group comprising spinal, knee, abdominal, hip, leg, arm, cranial, cardiovascular, facial and pelvic.

15. The patient positioning device as defined in claim 1 wherein said contoured patient support is operable to position the patient in a prone position.

16. The patient positioning device as defined in claim 3 wherein said coil array is formed integral within said contoured patient positioning device in a non-removable manner.

17. The patient positioning device as defined in claim 3 further comprising a coil array controller operable to control the driving of individual coils positioned within said coil array.

18. The patient positioning device as defined in claim 17 further comprising an imaging device operable to generate image data of the patient.

19. The patient positioning device as defined in claim 18 wherein said imaging device is selected from a group comprising a C-arm fluoroscopic imager, a magnetic resonance imager (MRI), a computed tomography (CT) imager, and a positron emission tomography (PET) imager, an isocentric fluoroscopy imager, a bi-plane fluoroscopy imager, an ultrasound imager, a multi-slice computed tomography (MSCT) imager, a high-frequency ultrasound (HIFU) imager, an optical coherence tomography (OCT) imager, an intra-vascular ultrasound imager (IVUS), a 2D, 3D or 4D ultrasound imager, an intra-operative CT imager, an intra-operative MRI imager, and a single photon emission computer tomography (SPECT) imager.

20. The patient positioning device as defined in claim 18 further comprising an instrument operable to be navigated by said electromagnetic navigation system and operable to be superimposed on said image data from said imaging device onto a display to navigate said instrument during the navigated medical procedure.

21. The patient positioning device as defined in claim 1 wherein said contoured patient support is radiolucent.

22. The patient positioning device as defined in claim 1 wherein said contoured patient support is formed of a support foam material.

23. The patient positioning device as defined in claim 22 wherein said support foam is overlaid on a support frame.

24. The patient positioning device as defined in claim 2 wherein said contoured patient support is substantially non-interfering with said electromagnetic navigation system.

25. The patient positioning device as defined in claim 1 wherein said contoured patient support is formed from an adjustable top of an OR table.

26. The patient positioning device as defined in claim 25 wherein said adjustable top includes at least one hinge to adjust an orientation of said top.

27. The patient positioning device as defined in claim 25 wherein said adjustable top is flexible.

28. A patient positioning system to position a patient during a navigated medical procedure, said patient positioning system comprising:
a first contoured patient support having a first shape and operable to position the patient with said first shape, said first contoured patient support defining a first recess;
a second contoured patient support having a second shape and operable to position the patient with said second shape, said second contoured patient support defining a second recess, wherein said first recess is substantially the same as said second recess, whereby a portion of a navigation system may be selectively received within each of said first and second recesses.

29. The patient positioning system as defined in claim 28 wherein said navigation system is an electromagnetic navigation system and said portion of said electromagnetic navigation system is a coil array.

30. The patient positioning system as defined in claim 29 wherein said coil array is positioned within a housing and said housing is removably received selectively within one of said first and second recesses.

31. The patient positioning system as defined in claim 28 wherein said first shape is different from said second shape.

32. The patient positioning system as defined in claim 30 wherein said housing is selected from a group of shapes comprising rectangular, triangular, round, square, hexagon, elliptical, octagon, U-shaped and polygon.

33. The patient positioning system as defined in claim 28 wherein said first and second recesses are defined by a cutout region on an underside of said first contoured patient support and said second contoured patient support, respectively.

34. The patient positioning system as defined in claim 28 wherein said first contoured patient support has a first size and said second contoured patient support has a second size, wherein said first shape is substantially the same as said second shape and said first size is different from said second size.

35. The patient positioning system as defined in claim 34 wherein said first shape and said second shape are a substantially arcuate shape.

36. The patient positioning system as defined in claim 29 further comprising a coil array controller operable to drive each coil in said coil array, an imaging device operable to obtain image date of the patient, and an instrument operable to be navigated with said electromagnetic navigation system and further operable to be superimposed on said image date and displayed on a display.

37. A patient positioning device to position a patient during a navigated medical procedure, said patient positioning device comprising:
means for supporting the patient in a contoured manner; and
means for receiving a portion of a navigation system within said means for supporting the patient, wherein the navigated medical procedure may be performed in a substantially unobstructed manner.

38. The patient positioning device as defined in claim 37 wherein said means for supporting the patient includes a support structure having a desired shape to support the patient in said contoured manner.

39. The patient positioning device as defined in claim 37 wherein said means for receiving a portion of a navigation system includes a recess formed in said means for supporting the patient.

40. The patient positioning device as defined in claim 39 wherein said recess removably receives said portion of said navigation system.

41. The patient positioning device as defined in claim 39 wherein said recess non-removably receives said portion of said navigation system.

42. The patient positioning device as defined in claim 37 wherein said portion of said navigation system is a coil array.

43. The patient positioning device as defined in claim 42 wherein said coil array is formed from three sets of orthogonal coils.

44. The patient positioning device as defined in claim 37 wherein said means for supporting the patient is removably positioned atop an operating table.

45. The patient positioning device as defined in claim 37 wherein said means for supporting the patient is formed integral with an operating table.

46. The patient positioning device as defined in claim 37 wherein said means for supporting the patient is attached to an operating table.

47. The patient positioning device as defined in claim 37 wherein said means for supporting the patient includes a U-shaped support clamp.

48. The patient positioning device as defined in claim 47 wherein said U-shaped support clamp includes said means for receiving a portion of the navigation system.
